(19) United States
(12) Patent Application Publication
(10) Pub. No.: US 2026/0174720 A1
(43) Pub. Date: Jun. 25, 2026

(54) NERVONIC ACID COMPOSITION FOR IMPROVING MEMORY-BASED LEARNING FUNCTION AND A PREPARATION METHOD THEREOF

(71) Applicant: Guangzhou Decade Nutrition Technology Co., Ltd., Guangzhou (CN)

(72) Inventor: Xingzhong Deng, Guangzhou (CN)

(21) Appl. No.: 19/070,620

(22) Filed: Mar. 5, 2025

(30) Foreign Application Priority Data
Mar. 5, 2024 (CN) 202410249639.7

(51) Int. Cl. A61K 31/202; A61K 9/00; A61K 38/00

(52) U.S. Cl. CPC A61K 31/202; A61K 9/0053; A61K 38/00; A61K 2300/00

(57) ABSTRACT

The present invention relates generally to the technical field of nutrition products, and more particularly, to a nervonic acid composition for improving memory-based learning function and a preparation method thereof. The present invention develops a multi-component and multi-efficacy nervonic acid composition, and the synergistic interaction of various nutrients contained in the composition gives the nervonic acid composition of the invention excellent effect of improving memory-based. learning function. The lysolecithin and resistant dextrin added in the preparation process of the nervonic acid composition can stabilize a variety of poorly stable and easily oxidized functional ingredients contained in the composition, including nervonic acid, DHA and arachidonic acid. The resulting nervonic acid composition has a longer shelf life and improved bioavailability of the functional ingredients in the composition.

### CROSS-REFERENCE TO RELATED APPLICATIONS

This is a U. S. patent application which claims the priority and benefit of Chinese Patent Application Number 202410249639.7, filed on Mar. 5, 2024, the disclosure of which is incorporated herein by reference in its entirety.

### TECHNICAL FIELD

The present invention relates generally to the technical field of nutrition products, and more particularly, to a nervonic acid composition for improving memory-based learning function and a preparation method thereof.

### BACKGROUND

Nervonic Acid (NA) is a type of @-9 long-chain unsaturated monoleic fatty acid, also known as Selacholeic Acid, with the chemical name of cis-15-Tetracosenic Acid, molecular formula of C24H4602 and the molecular weight of 366.6. Nervonic acid is white flake crystal at room temperature, insoluble in water, soluble in alcohols such as methanol, ethanol, etc., with a melting point of 41-43° C. Nervonic acid was first found in the nervous tissues of mammals. It is an important component of the biofilm in the nervous tissues, and the characteristic ingredient of the medullary brain glycosides. It is mainly derived from shark brain and shark oil. The process of extracting nervonic acid from natural plants or by fermentation has also been developed.

Nervonic acid can completely pass through the blood-brain barrier and directly act on nerve fibers for repair and dredging to regenerate the damaged and shedding protecting sheath, dissolve the necrotic tissues blocking the channels, and induce the self-growth and division of nerve fibers, so that the information generated by nerve cells and external information can be smoothly transmitted through nerve fibers to achieve unblocked commands. In this way, the damaged, diseased and dormant nerve cells are activated, the neural network is reshaped, and some or all functions of patients in language, memory, sensation, limb and other aspects are restored to achieve complete rehabilitation of encephalopathy.

Because of the good physiological activity and difficulty in generating by the human body itself, the nervonic acid can only be supplemented by in vitro intake, so it has become a new generation of health additives highly favored at home and abroad.

CN109288005A discloses a composition with auxiliary memory improvement function, which combines folic acid compounds, algal oil DHA powder, nervonic acid and N-acetylneuramic acid, and controls the weight ratio of various components, so that the synergistic effect between different components can effectively improve the reversible lapse of memory.

CN110200288A discloses an infant nutrition composition, which contains zinc, vitamin D, nervonic acid and oil substances, which can increase the content of nervonic acid in the infant plasma to meet the needs of infants for nervonic acid, zinc and vitamin D, significantly improve the memory, promote the healthy development of infant nervous system, and further improve the intestinal immune resistance by promoting the transformation of immune cells.

Despite the above studies, on the one hand, the composition disclosed in these studies has simple ingredients, relatively single functions, and relatively general improvement effect on the memory-based learning function of infants; on the other hand, both nervonic acid and DHA have poor stability and easy oxidation, resulting in a short shelf life.

### SUMMARY

In order to solve the above problems, the present invention develops a multi-component and multi-efficacy nervonic acid composition with excellent effect of improving memory-based learning function. The present invention also unexpectedly finds that by adding lysolecithin and resistant dextrin in the preparation process of the nervonic acid composition, the resulting nervonic acid composition has a longer shelf life and improved bioavailability.

Specifically, the present invention involves a nervonic acid composition for improving memory-based learning function, comprising 20-25 parts of nervonic acid, 7-9 parts of DHA, 1.5-2.5 parts of phosphatidylserine, 4-5 parts of arachidonic acid, 0.01-1 parts of walnut peptide, 6-7 parts of n-acetylneuroaminic acid, 0.02-1 parts of taurine, 5-6 parts of isomaltooligosaccharide, 0.02-0.05 parts of lactase, 0.5-1.2 parts of peony seed oil peptide, 1-2 parts of L-theanine, 0.5-1 parts of magnesium, 0.05-0.1 parts of iron, 0.03-0.1 parts of zinc, 0.01-0.02 parts of vitamin B6, 18-20 parts of lysolecithin and 16-20 parts of resistant dextrin by weight.

Furthermore, the said nervonic acid is derived from maple seed oil.

Furthermore, the said nervonic acid composition is in the form of solid powder or emulsion.

Furthermore, the said magnesium exists in the form of magnesium sulfate or magnesium lactate.

Furthermore, the said iron exists in the form of ferrous sulfate or ferrous lactate.

Furthermore, the said zinc exists in the form of zinc gluconate, zinc citrate or zinc lactate.

The present invention also involves a method for preparing a nervonic acid composition as described herein, comprising:
(1) Add lysolecithin and resistant dextrin to deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen or carbon dioxide from the bottom of the emulsion tank, emulsify at 50-60° C. for 10-30 min under stirring and cool to room temperature to obtain an emulsion after homogenization.
(4) The said nervonic acid composition is obtained by vacuum concentration and sterilization of the emulsion of step (3), or by spray drying and sterilization of the emulsion of step (3) with nitrogen or carbon dioxide as the drying medium.

Furthermore, the amount of deionized water used in step (1) is 5 times the mass of lysolecithin and resistant dextrin.

Furthermore, the said magnesium-containing substances include magnesium sulfate or magnesium lactate.

Furthermore, the said iron-containing substances include ferrous sulfate or ferrous lactate.

Furthermore, the said zinc-containing substances include zinc gluconate, zinc citrate or zinc lactate.

Furthermore, the said stirring speed in step (3) is 2000-5000 r/min.

Furthermore, the said homogenization in step (3) includes filtering the emulsified liquid through a 100-mesh sieve and homogenizing the filtrate at 50° C. and 15 MPa for 10-30 min.

Furthermore, the said spray drying in step (3) has the inlet air temperature of 100-200° C. and the outlet air temperature of 30-80° C.

Furthermore, the said sterilization in step (4) includes pasteurization at 80-85° C. for 10-15 min.

The present invention also involves the application of a nervonic acid composition in formula milk powder as described herein.

Furthermore, the additive amount of nervonic acid composition in formula milk powder as described herein is 2-3 wt %.

### Beneficial Effect of the Present Invention

The present invention develops a multi-component and multi-efficacy nervonic acid composition, and the synergistic interaction of various nutrients contained in the composition gives the nervonic acid composition of the invention excellent effect of improving memory-based. learning function. In addition, the present invention also unexpectedly finds that the lysolecithin and resistant dextrin added in the preparation process of the nervonic acid composition can stabilize a variety of poorly stable and easily oxidized functional ingredients contained in the composition, including nervonic acid, DHA and arachidonic acid. The resulting nervonic acid composition has a longer shelf life and, further, improved bioavailability of the functional ingredients in the composition.

### DESCRIPTION OF EMBODIMENTS

The embodiments described below are used to further explain the present invention, rather than to limit the present invention in any form. Unless otherwise specified, the reagents, methods and equipment used in the present invention are conventional reagents, methods and equipment in the field of the invention.

### Embodiment 1

This embodiment involves a nervonic acid composition for improving memory-based learning function, comprising 20 parts of nervonic acid, 9 parts of DHA, 2 parts of phosphatidylserine, 5 parts of arachidonic acid, 1 part of walnut peptide, 7 parts of n-acetylneuroaminic acid, 1 part of taurine, 6 parts of isomaltooligosaccharide, 0.05 parts of lactase, 1 part of peony seed oil peptide, 2 parts of L-theanine, 1 part of magnesium, 0.1 parts of iron, 0.1 parts of zinc, 0.02 parts of vitamin B6, 18 parts of lysolecithin and 16 parts of resistant dextrin by weight.

The nervonic acid composition is prepared as follows:
(1) Add lysolecithin and resistant dextrin to 5 times the weight of deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen from the bottom of the emulsion tank, emulsify at 60° C. for 10-30 min under stirring at 2500 r/min, cool to room temperature, filter the emulsified liquid through a 100-mesh sieve and homogenize the filtrate at 50° C. and 15 MPa for 30 min to obtain an emulsion.
(4) Spray dry the emulsion of step (3) with nitrogen as the drying medium, at the inlet air temperature of 120° C., and the outlet air temperature of 45° C., then pasteurize the resulting product at 85° C. for 15 min to obtain the said nervonic acid composition in solid powder form.

### Embodiment 2

This embodiment involves a nervonic acid composition for improving memory-based learning function, comprising 25 parts of nervonic acid, 7 parts of DHA, 2 parts of phosphatidylserine, 4 parts of arachidonic acid, 1 part of walnut peptide, 6 parts of n-acetylneuroaminic acid, 0.05 parts of taurine, 5 parts of isomaltooligosaccharide, 0.05 parts of lactase, 1 part of peony seed oil peptide, 1 part of L-theanine, 0.5 parts of magnesium, 0.05 parts of iron, 0.05 parts of zinc, 0.01 parts of vitamin B6, 20 parts of lysolecithin and 18 parts of resistant dextrin by weight.

The nervonic acid composition is prepared as follows:
(1) Add lysolecithin and resistant dextrin to 5 times the weight of deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen from the bottom of the emulsion tank, emulsify at 60° C. for 30 min under stirring at 3500 r/min, cool to room temperature, filter the emulsified liquid through a 100-mesh sieve and homogenize the filtrate at 50° C. and 15 MPa for 30 min to obtain an emulsion.
(4) Concentrate the emulsion of step (3) under vacuum and then pasteurize it at 85° C. for 15 min to obtain the said nervonic acid composition in emulsion form.

### Comparative Example 1

This comparative example a nervonic acid composition for improving memory-based learning function, which differs from Embodiment 1 only in that the lysolecithin is replaced with lecithin.

### Comparative Example 2

This comparative example a nervonic acid composition for improving memory-based learning function, which differs from Embodiment 1 only in that the lysolecithin is replaced with lecithin and the additive amount of lecithin is 30 parts by weight.

### Comparative Example 3

This comparative example a nervonic acid composition for improving memory-based learning function, which differs from Embodiment 1 only in that the lysolecithin is replaced with lecithin and the additive amount of lecithin is 60 parts by weight.

### Comparative Example 4

This comparative example a nervonic acid composition for improving memory-based learning function, which differs from Embodiment 1 only in that it does not contain lysolecithin and resistant dextrin.

### Test Example 1: Oxidation Stability Analysis of Nervonic Acid Composition

The nervonic acid compositions prepared in Embodiments 1-2 and comparative examples 1˜4 were placed in a constant temperature oven at 60° C., and sampled every 12 hours to test the peroxide value using the oil oxidation stability analyzer by the titration method specified in GB5009.227-2016. Each sample was tested three times and the average value was obtained, as shown in Table 1.

TABLE 1

| Accelerated | | | | | | |
|---|---|---|---|---|---|---|
| oxidation | Embodiment | Embodiment | Comparative | Comparative | Comparative | Comparative |
| time (h) | 1 | 2 | example 1 | example 2 | example 3 | example 4 |
| 0 | 0.00068 | 0.00069 | 0.00075 | 0.00072 | 0.00070 | 0.00081 |
| 12 | 0.00489 | 0.00524 | 0.00752 | 0.00604 | 0.00512 | 0.02689 |
| 24 | 0.01016 | 0.01129 | 0.01739 | 0.01286 | 0.01039 | 0.04876 |
| 36 | 0.02832 | 0.02992 | 0.03941 | 0.03392 | 0.03021 | 0.10385 |
| 48 | 0.04628 | 0.04923 | 0.11832 | 0.09873 | 0.05538 | 0.19652 |
| 60 | 0.10692 | 0.11271 | 0.20467 | 0.16492 | 0.12084 | 0.26847 |
| 72 | 0.12683 | 0.13552 | 0.25031 | 0.21793 | 0.14965 | 0.29751 |

As can be seen from the results in Table 1, compared with the comparative example 4 that does not contain lysolecithin and resistant dextrin, both the embodiments 1-2 and comparative examples 1-3 can significantly inhibit the growth of peroxide value and have better oxidation stability. Among them, embodiments 1-2 have higher stability. However, replacing lysolecithin with lecithin can also enhance oxidation stability, but requires a larger amount (60 parts by weight) of lecithin to achieve the oxidation stability close to embodiments 1-2. Such a large amount of lecithin added is obviously not in conformity with relevant industry standards when the nervonic acid composition of the present invention is applied to formula milk powder.

### Test Example 2: Bioavailability Test

The nervonic acid compositions prepared in embodiments 1-2 and comparative examples 1˜4 were added to skim milk powder free of any additives under sterile conditions at 3 wt %, respectively, for later use.

A total of 70 volunteers were selected for the human trial on improving memory. The volunteers were of the same age group (16-18 years old) and had the same education level (sophomore in high school), who had not received similar experiments and not taken drugs and health products related to improving memory. The volunteers were divided into seven groups with 10 people in each group. Each group had basically the same gender composition, mean memory quotient and standard deviation. One group was randomly selected as the control group, and the others were experimental groups. The volunteers were treated by double-blind method. The experimental groups were given degreased skim milk powder prepared by nervonic acid compositions from embodiments 1-2 and comparative examples 1-4, respectively, while the control group was given skim milk powder free of any additives, for one consecutive month at 25 g/60 kg per day. The test was carried out according to the Wechsler Intelligence Scale-Revised, to test the mind, recognition, association, picture and understanding. The results are shown in Table 2.

TABLE 2

| | | | Comparative | Comparative | Comparative | Comparative | |
|---|---|---|---|---|---|---|---|
| Test | Embodiment | Embodiment | example | example | example | example | Control |
| item | 1 | 2 | 1 | 2 | 3 | 4 | group |
| Mind | 31.34 ± | 32.11 ± | 31.56 ± | 30.94 ± | 32.54 ± | 31.23 ± | 31.51 ± |
| | 4.22 | 4.17 | 4.31 | 4.74 | 4.01 | 4.43 | 4.25 |
| Recognition | 17.53 ± | 17.28 ± | 14.59 ± | 15.23 ± | 16.96 ± | 14.11 ± | 9.36 ± |
| | 3.67 | 3.94 | 2.05 | 2.62 | 3.81 | 2.89 | 2.52 |
| Lenovo | 16.15 ± | 16.05 ± | 15.59± | 15.72 ± | 16.01 ± | 15.16 ± | 9.85 ± |
| | 2.46 | 2.68 | 2.88 | 2.34 | 2.92 | 2.02 | 2.61 |
| Picture | 16.83 ± | 16.89 ± | 15.52 ± | 15.91 ± | 16.53 ± | 15.13 ± | 8.97 ± |
| | 1.86 | 2.04 | 1.42 | 1.25 | 1.33 | 2.03 | 2.31 |
| Understand | 18.34 ± | 18.12 ± | 15.96 ± | 16.77 ± | 17.96 ± | 15.60 ± | 9.12 ± |
| | 2.23 | 2.68 | 1.86 | 1.69 | 2.52 | 2.34 | 2.02 |

As can be seen from the results in Table 2, the volunteers who took the skim milk powder prepared by nervonic acid compositions from embodiments 1-2 and comparative examples 1˜4 continuously for 30 days had improved test scores of recognition, association, picture and understanding compared with the control group, indicating that the nervonic acid composition prepared in this application can effectively help improve the memory of volunteers by adding a variety of active ingredients and controlling their content and mass ratio, and that the volunteers have no adverse reactions during the administration. At the same time, it can also be seen that, compared with the comparative example 4 that does not contain lysolecithin and resistant dextrin, the volunteers who took the skim milk powder prepared by nervonic acid compositions from embodiments 1-2 and comparative examples 1-3 continuously had improved test scores of recognition, association, picture and understanding, indicating that the addition of lysolecithin and resistant dextrin can improve the bioavailability of functional ingredients in the composition.

It should be noted that the specification of the present invention gives better embodiments of the invention. However, the present invention can be embodied in many different forms, not limited to the embodiments described in the specification. These embodiments are not intended as additional limitations on the contents of the present invention and are provided for the purpose of providing a more thorough and comprehensive understanding of the disclosed content of the invention. In addition, the above technical features continue to combine with each other to form various embodiments not listed above, which are deemed to fall in the scope of the specification of the present invention; furthermore, improvements or transformations may be made by persons of ordinary skill in the art in accordance with the above description, and all such improvements and transformations shall fall within the scope of protection of the claims attached to the present invention.

What is claimed is:

1. A nervonic acid composition for improving memory-based learning function, wherein the composition comprises 20-25 parts of nervonic acid, 7-9 parts of DHA, 1.5-2.5 parts of phosphatidylserine, 4-5 parts of arachidonic acid, 0.01-1 parts of walnut peptide, 6-7 parts of n-acetylneuroaminic acid, 0.02-1 parts of taurine, 5-6 parts of isomaltooligosaccharide, 0.02-0.05 parts of lactase, 0.5-1.2 parts of peony seed oil peptide, 1-2 parts of L-theanine, 0.5-1 parts of magnesium, 0.05-0.1 parts of iron, 0.03-0.1 parts of zinc, 0.01-0.02 parts of vitamin B6, 18-20 parts of lysolecithin and 16-20 parts of resistant dextrin by weight;
The method for preparing the nervonic acid composition comprises:
(1) Add lysolecithin and resistant dextrin to deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen or carbon dioxide from the bottom of the emulsion tank, emulsify at 50-60° C. for 10-30 min under stirring and cool to room temperature to obtain an emulsion after homogenization.
(4) The said nervonic acid composition is obtained by vacuum concentration and sterilization of the emulsion of step (3), or by spray drying and sterilization of the emulsion of step (3) with nitrogen or carbon dioxide as the drying medium.

2. The nervonic acid composition according to claim 1, wherein the said nervonic acid composition is in the form of solid powder or emulsion.

3. A method for preparing any said nervonic acid composition according to claim 1, wherein the method comprises:
(1) Add lysolecithin and resistant dextrin to deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen or carbon dioxide from the bottom of the emulsion tank, emulsify at 50-60° C. for 10-30 min under stirring and cool to room temperature to obtain an emulsion after homogenization.
(4) The said nervonic acid composition is obtained by vacuum concentration and sterilization of the emulsion of step (3), or by spray drying and sterilization of the emulsion of step (3) with nitrogen or carbon dioxide as the drying medium.

4. A method for preparing any said nervonic acid composition according to claim 2, wherein the method comprises:
(1) Add lysolecithin and resistant dextrin to deionized water and stir until evenly dispersed.
(2) Uniformly mix the solid powder of nervonic acid, DHA, phosphatidylserine, arachidonic acid, walnut peptide, n-acetylneuroaminic acid, taurine, isomaltooligosaccharide, lactase, peony seed oil peptide, L-theanine, magnesium-containing substances, iron-containing substances, zinc-containing substances and vitamin B6, then add to the mixture of step (1), and continue stirring until evenly dispersed.
(3) Place the mixture of step (2) in an emulsion tank, continuously introduce nitrogen or carbon dioxide from the bottom of the emulsion tank, emulsify at 50-60° C. for 10-30 min under stirring and cool to room temperature to obtain an emulsion after homogenization.
(4) The said nervonic acid composition is obtained by vacuum concentration and sterilization of the emulsion of step (3), or by spray drying and sterilization of the emulsion of step (3) with nitrogen or carbon dioxide as the drying medium.

5. The method according to claim 3, wherein the amount of deionized water used in step (1) is 5 times the mass of lysolecithin and resistant dextrin.

6. The method according to claim 4, wherein the amount of deionized water used in step (1) is 5 times the mass of lysolecithin and resistant dextrin.

7. The method according to claim 3, wherein the said magnesium-containing substances include magnesium sulfate or magnesium lactate, the said iron-containing substances include ferrous sulfate or ferrous lactate and the said zinc-containing substances include zinc gluconate, zinc citrate or zinc lactate.

8. The method according to claim 4, wherein the said magnesium-containing substances include magnesium sulfate or magnesium lactate, the said iron-containing substances include ferrous sulfate or ferrous lactate and the said zinc-containing substances include zinc gluconate, zinc citrate or zinc lactate.

9. The method according to claim 3, wherein the said stirring speed in step (3) is 2000-5000 r/min.

10. The method according to claim 4, wherein the said stirring speed in step (3) is 2000-5000 r/min.

11. The method according to claim 3, wherein the said homogenization in step (3) includes filtering the emulsified liquid through a 100-mesh sieve and homogenizing the filtrate at 50° C. and 15 MPa for 10-30 min.

12. The method according to claim 4, wherein the said homogenization in step (3) includes filtering the emulsified liquid through a 100-mesh sieve and homogenizing the filtrate at 50° C. and 15 MPa for 10-30 min.

13. The method according to claim 3, wherein the said spray drying in step (3) has the inlet air temperature of 100-200° C. and the outlet air temperature of 30-80° C.

14. The method according to claim 4, wherein the said spray drying in step (3) has the inlet air temperature of 100-200° C. and the outlet air temperature of 30-80° C.

15. An application of a nervonic acid composition in formula milk powder according to claim 1.

16. An application of a nervonic acid composition in formula milk powder according to claim 2.

17. The application according to claim 15, wherein the additive amount of nervonic acid composition in formula milk powder is 2-3 wt %.

18. The application according to claim 16, wherein the additive amount of nervonic acid composition in formula milk powder is 2-3 wt %.
